(19) United States
(12) Patent Application Publication
(10) Pub. No.: US 2025/0241884 A1
(43) Pub. Date: Jul. 31, 2025

(54) Pharmaceutical Compositions of D10-Docosahexaenoic Acid or Esters Thereof

(71) Applicant: BioJiva LLC, San Jose, CA (US)

(72) Inventors: Karsten Schmidt, La Jolla, CA (US); Mikhail Shchepinov, Oxford (GB)

(21) Appl. No.: 19/072,731

(22) Filed: Mar. 6, 2025

Related U.S. Application Data
(63) Continuation of application No. 18/167,783, filed on Feb. 10, 2023, now abandoned.
(60) Provisional application No. 63/403,670, filed on Sep. 2, 2022.

(51) Int. Cl. A61K 31/202; A61P 27/02; G01N 33/92

(52) U.S. Cl. CPC A61K 31/202; A61P 27/02; G01N 33/92

(57) ABSTRACT

Disclosed are methods for assessing the presence or absence of a therapeutic concentration of a deuterated docosahexaenoic acid during treatment of a patient with a retinal or neuronal oxidative disease.

### CROSS-REFERENCE

This application is a continuation of U.S. application Ser. No. 18/167,783, filed Feb. 10, 2023, which claims the benefit of U.S. Provisional Application No. 63/403,670, filed Sep. 2, 2022, which is incorporated by reference herein in its entirety.

### FIELD

Disclosed are pharmaceutical compositions comprising from about 100 mg to about 1,250 mg of D-10-docosahexaenoic acid and, preferably, about 250 mg, or about 500 mg, or about 1,000 mg of D-10-docosahexaenoic acid. The D-10-docosahexaenoic acid can be delivered either as an acid (“active”), or as an ester, or as a pharmaceutically acceptable salt thereof (collectively “D-DHA” unless the context dictates otherwise). These compositions are useful for treating oxidative diseases mediated, at least in part, by lipid peroxidation including, for example, neuronal and retinal oxidative diseases. The amount of active in these compositions is set to achieve an in vivo therapeutic steady state concentration of the D-DHA (acid) independent of the amount of docosahexaenoic acid (DHA) consumed by the patient.

### BACKGROUND

DHA is an essential fatty acid. The primary source of DHA is from food and especially oily seafood which is particularly rich in DHA. On average, about 90% of adults over 50 years of age in the United States consume food containing between 70 milligrams (mean) and 130 milligrams (90% level) of docosahexaenoic acid (DHA) per day; only 10% of adults exceed daily intakes of 130 milligrams (mg) (Papanikolaou, Nutrition Journal, 13:31-37 (2014) which is incorporated herein by reference in its entirety). Only 5% of the same population consumes more than 180 mg DHA per day, i.e. the 95^{th} percentile increases the daily consumption of DHA to up to about 180 mg/day of DHA (USDA, Agricultural Research Service, 2021 “Usual Nutrient Intake from Food and Beverages, by Gender and Age, What We Eat in America—http://www.ars.usda.gov/nea/bhnrc/fsrg) which is also incorporated herein by reference in its entirety. As is well known, DHA is the major structural polyunsaturated fatty acid component in phospholipid membranes of neurons and of rods and cones of the retina.

Peroxidation of DHA, including that occurring in neurons or the retina, accounts for a number of devastating degenerative diseases. For example, retinal oxidative diseases include, but are not limited to, wet and dry age-related macular degeneration (including geographic atrophy associated therewith), retinal degeneration, cataracts, retinitis pigmentosa, diabetic retinopathy, glaucoma, and Stargardt Disease. Such neuronal oxidative diseases include, but are not limited to, Amyotrophic Lateral Sclerosis (ALS), Jacobson Syndrome, spinal muscular atrophy, multiple system atrophy, Alzheimer's Disease (AD) and mild cognitive impairment, Huntington's Disease, infantile neuroxonal disease (INAD), Parkinson's Disease, Progressive supranuclear palsy (PSP) to name a few.

The use of certain deuterated polyunsaturated fatty acids or esters thereof to treat these diseases is disclosed in U.S. Pat. No. 10,058,522 which is incorporated herein by reference in its entirety. While the underlying etiology of these diseases are different, the subsequent pathologies all include lipid peroxidation of polyunsaturated fatty acids (PUFAs) such as DHA where oxidation initiates at one or more of the bis-allylic positions of these PUFAs. Once initiated, the first oxidized site then initiates oxidation of adjacent sites on neighboring PUFAs in a chain-reaction which process is defined as lipid peroxidation.

Treating oxidative diseases, including retinal and neuronal oxidative diseases, with D-DHA is complicated by the fact that it can take weeks to a few months after the initiation of treatment to reach a therapeutic concentration at the target sites in the body (e.g., neurons or retina). Moreover, once such a therapeutic concentration is achieved, patients are required to continue treatment for the balance of their lives in order to maintain that concentration to treat the underlying chronic disease condition.

Still further, unlike most therapeutic agents, D-DHA as a drug is meant to supplement a naturally occurring essential fatty acid whereas most therapeutic agents do not have a naturally occurring counterpart. This means that the clinician needs to balance the amount of D-DHA delivered to the patient to achieve a therapeutically effective ratio between the amount of D-DHA incorporated into targeted sites in vivo such as neurons and the retina with the amount of naturally occurring DHA. Balancing such a dichotomy is complicated by at least four significant factors: 1) variable patient consumption levels of naturally occurring DHA, 2) patient medication non-compliance, 3) determining the relative proportion of the amount of naturally occurring DHA and D-DHA in the patient at any given time in order to achieve a therapeutic concentration of D-DHA in the target tissue or cells, and 4) confirming that such a therapeutic concentration is maintained.

Due to dilution by dietary consumption of natural DHA, the relative percentage of a fixed amount of deuterated DHA (D-DHA) as it relates to the total amount of DHA including D-DHA is not 100%. This means that at a fixed dose of D-DHA, the percent amount of D-DHA absorbed by the patient and incorporated into the body is controlled by the total pool of DHA consumed by the patient. Stated differently, the more naturally occurring DHA consumed during therapy, the more it reduces the relative percentage of a fixed dose of D-DHA administered and, accordingly, the percentage of D-DHA that is absorbed and incorporated into the body is dependent on the amount of DHA consumed. Still further, oily seafood and fish oil contain high levels of natural DHA. For patients whose diets including significant amounts of such seafood and/or for those who use fish oil based medicaments or dietary supplements, the increased amount of DHA becomes a problem as it significantly increases the average daily amount of DHA consumed. As above, this will reduce the relative proportion of a fixed dosed of D-DHA that is consumed and then incorporated as a pool of total DHA into the body.

Still further, apart from food frequency questionaires that determine average intakes in large populations, there is no reliable direct method that can determine the average amount of DHA consumed by an individual patient prior to initiating treatment. So, for patients initiating therapy with D-DHA, the clinician can only approximate the amount of DHA consumed in their diets based on a detailed food questionnaire analysis of their historical diet. While such maybe generally predictable for some patients, that analysis will not be predictable for all patients most likely because patients typically provide a poor recollection of their diet including both content and quantity. Moreover, the diet for any given patient varies from day-to-day and from time-to-time which renders the average amount of consumed DHA in the patient as a continuously moving variable which adds a further level of complexity and unpredictability. Accordingly, results that evidence that the drug is ineffective for that patient can be tied to the patient's consumption of too much DHA or to medication non-compliance or both. Regardless, the results for that patient may be that D-DHA was ineffective whereas, the failure was not due to the drug but to factors unrelated thereto.

As to patient compliance, it is well known that the lack of medication compliance (missing multiple days of medication per month) for patients with chronic diseases occurs in about 50% or more of treated patients. See, for example, uspharmacist.com/article/medication-adherence-the-elephant-in-the-room which is incorporated herein by reference in its entirety. This decreases the apparent efficacy of the drug and potentially renders an otherwise beneficial drug as being considered insufficiently efficacious. Currently, medication non-compliance during therapy for chronic diseases is a serious problem.

Finally, determining a reliable moving average of the concentration of DHA in vivo for a given patient and then to balance that with the requisite amount of D-DHA to be administered to achieve a therapeutic concentration in, e.g., neurons and the retina, is exceptionally difficult. This is because the number of variables set forth above and particularly the unavailability of assays to assess the amount of DHA found in a blood sample and also because neurons and the retina are inaccessible in living patients. As above, the amount of active D-DHA absorbed into a patient at a fixed dose of D-DHA is dependent on the amount of DHA in that patient's diet. Stated in other terms, the more DHA consumed, the lower the active D-DHA proportion that reaches the target tissue. So, at the start of therapy, the drug developer and clinician is relying on the published statistical average data in the target population, but is unable to determine if the dose of D-DHA provided to a patient is always sufficient to treat the condition effectively when administered chronically.

Accordingly, there is an urgent need to provide for a pharmaceutical composition that obviates the amount of DHA consumed by a patient as a factor in whether D-DHA is efficacious, reduces the effect of medication non-compliance on the validity of the results obtained, and then provide for means to test for the relative ratio of active D-DHA to DHA in the treated patient to assure that the patient achieves and maintains a therapeutic level of active D-DHA.

### SUMMARY

Disclosed are pharmaceutical compositions comprising a therapeutically effective amount of D-DHA and methods related thereto where the average amount of DHA consumed by patients is unrelated to the dose of D-DHA initially provided to the patient per day. As a result, variations in the amount of DHA consumed is eliminated in all patients at the start of therapy.

Also disclosed are methods for analyzing the ratio of active D-DHA in a patient relative to the total amount of DHA present in the patient's blood. These methods are conducted after the start of therapy and preferably the initial analysis is conducted about 14 to 45 days after the start of therapy (plasma steady state concentrations of active D-DHA is reached in about 14 days after start of therapy and in about 45 days red blood cell). In these methods, about 14 to 45 days after the start of therapy, a clinician can determine the in vivo concentration of both active D-DHA and DHA by a minimally invasively analysis of the patient's plasma or red blood cells. Specifically, knowing what the daily dose of D-DHA administered to patients, allows the clinician to determine the amount of active D-DHA in the blood (plasma or red blood cells). In turn, correlating that amount to the relative amount of DHA to the known amount of active D-DHA, the clinician can then extrapolate the in vivo average amount of DHA consumed by the patient. When combined, the attending clinician can adjust the amount of D-DHA in the pharmaceutical compositions as needed or ask the patient to adjust the dietary habit (e.g., by reducing the intake of oily fish consumed by the patient). Accordingly, the compositions and methods provided herein are able to treat patients with therapeutic doses of D-DHA independent of the amount of DHA in the patient at the start of therapy. Moreover, periodic analysis of the ratio of active D-DHA to DHA in the patient's blood allows the clinician to determine if the treated patient is maintaining a therapeutic ratio of active D-DHA to DHA. If not, then the clinician can either increase the dose of D-DHA, administer a bolus injection of D-DHA, or instruct the patient to reduce the amount DHA, including oily fish, consumed or to be medication compliant. Still further, the clinician can then reevaluate all patients at subsequent points in time to ensure that all are maintaining a therapeutic ratio of active D-DHA to DHA.

In addition, when a steady-state concentration of active D-DHA is achieved in vivo in major organs and tissues, such as in the skeletal muscle and the liver, the amount of D-DHA in the disclosed pharmaceutical compositions can be adjusted to provide for several days to weeks of medication non-compliance per month as summarized in Tables 5-7.

The compositions and methods disclosed herein are based on two specific molecular features of D-DHA: (i) D-DHA has a higher molecular mass than DHA which allows quantitative analysis of D-DHA in the presence of natural DHA with standard analytical methods, and (ii) active D-DHA and DHA have the same absorption, distribution, and elimination kinetics. They involve multiple factors that are either fixed or variable for a given patient and/or vary from patient to patient including the following:
the ability to analyze each patient's blood to determine the overall concentration of DHA (including active D-DHA) as well as the ratio of active D-DHA to DHA;
correlating whether a patient's average consumption of DHA provides for a therapeutic ratio of active D-DHA to DHA for a given dose of D-DHA recognizing the variability in DHA consumption for that patient can vary day-to-day, week-to-week and season-to-season;
identifying that portion of the treated patients who do not achieve a therapeutic concentration due to consuming too much DHA or who are medication non-compliant;
the ability to identify patients that consume above average (i.e. multiple) oily seafood meals per week;
determining a therapeutic in vivo ratio of active D-DHA to DHA and then correlating that ratio to the daily dose D-DHA while allowing for a wide variability in the average amount of DHA per day consumed by the patient; and
recognizing a level of medication non-compliance and how to address the same while maintaining an efficacious therapeutic result.

Heretofore, it was not possible to determine the average daily consumption of DHA directly because there was no internal standard to measure it by. Stated differently, a clinician could quantify the amount of DHA in the blood but not its precise correlation to daily dietary intakes because a portion of DHA present in body tissues is constantly reycled in the human body and redistributed via the blood circulation. This invention is based, in part, on the discovery that D-DHA has a dual function as a therapeutic agent and at the same time as an internal standard that is distinguishable from natural DHA by its molecular mass. Therefore, initially dosing a patient with a fixed dose of D-DHA, based on the statistical average of the average daily DHA consumption in the target patient population, allows for the clinician to assess the patient's blood concentration of both actove D-DHA and DHA after a steady state concentration of active D-DHA is reached in blood (i.e., plasma or red blood cells). Moreover, when the concentration of active D-DHA in the plasma or red blood cells of a patient reaches steady state, the determination of the amount of active D-DHA in a blood sample allows a clinician to determine the average amount of DHA consumed per day by that patient based on the known daily dose of D-DHA administered.

In one aspect, there is provided a method for assessing the average daily consumption amount of DHA in a patient comprising:
a) administering a fixed daily amount of D-DHA to the patient for a period of time sufficient to reach steady state concentrations in the patient's blood or a component thereof;
b) obtaining a sample of the patient's blood or a component thereof at successive time intervals and assessing the amount of active D-DHA in thesample based on the fixed daily amount of D-DHA administered as well as the ratio of active D-DHA to DHA; and
c) correlating the ratio of active D-DHA to DHA with the amount of active D-DHA found in the blood or a component thereof to obtain a value of DHA in the blood over time.

In some embodiments, the blood component tested is plasma and the fixed daily amount of D-DHA administered to the patient reaches a steady state concentration in the plasma within about 14 days.

In some embodiments, the blood component tested are red blood cells and the fixed daily amount of D-DHA administered to the patient reaches a steady state concentration in these cells within about 45 days.

As evidenced in the examples below, in vivo data evidence that a concentration of at least about 1:4 active D-DHA to DHA (at least about 20% of the total amount of DHA in the plasma is active D-DHA) is sufficient to provide for therapeutic reduction in lipid peroxidation and, preferably, a ratio of at least about 1:1 active D-DHA to DHA near maximal results are obtained. Based on the average daily amount of DHA consumed by the patient and the concentration of active D-DHA in the patient's blood relative to the entire amount of DHA (including active D-DHA), the clinician can retain the same dose of D-DHA or adjust (increase/decrease) the dose to arrive at an in vivo target ratio of active D-DHA to DHA to provide for therapeutic results for that patient.

Because D-DHA is well tolerated with minimal adverse events, concentrations up to or exceeding 1,250 mg/day can be used. This allows for dosing regimens to employ from about 50 mg/day up to about 1,250 mg/day or higher or from 100 mg/day up to 1,000 mg/day. As there is considerably variability in a diet over time and given the large therapeutic window for D-DHA, the target ratio of active D-DHA to DHA is preferably set at least about 1:3, or at least about 2:3, or at least about 1:1, or at least about 2:1 or higher in order to account for such variability provided that the preferred maximum dose of D-DHA is no more than 1,250 mg/day.

In another aspect, there is provided a method for treating an oxidative neurodegenerative or retinal disease in a patient comprising:
a) administering a fixed daily amount of D-DHA to the patient for a period of time sufficient to reach steady state concentrations in the patient's blood or a component thereof;
b) obtaining a sample of the patient's blood or a component thereof at successive time intervals and assessing the amount of active D-DHA in the sample based on the fixed daily amount of D-DHA administered as well as the ratio of active D-DHA to DHA;
c) correlating the ratio of active D-DHA to DHA with the amount of active D-DHA found in the blood or a component thereof to obtain a value of DHA in the blood over time;
d) adjusting the amount of the fixed daily amount of D-DHA as necessary to achieve a steady state concentration of active D-DHA to DHA of at least about 1:4 in the patient; and
e) continuing the adjusted fixed daily amount of D-DHA in order to treat the disease.

In some embodiments, patients being treated with D-DHA are scheduled to have periodic blood samples analyzed to confirm that the target ratio of active D-DHA to DHA is being maintained. Such periodic blood draws can be conducted monthly, quarterly, semi-annually, annually or at any interval deemed appropriate by the attending clinician. In addition, confirming that maintenance of the targeted active D-DHA to DHA ratio, such periodic testing can assess whether the daily amount of DHA being consumed remains substantially the same (+/−20%) as found in previous tests or whether the patient is being medication compliant.

In some embodiments, the adjusted fixed daily dose of D-DHA is set to achieve a steady state concentration in blood of active D-DHA to DHA of about 1:3, or about 2:3, or about 1:1 or about 2:1 or higher. In a preferred embodiment, the fixed daily dose of D-DHA assigned by the clinician at the start of therapy is set at a concentration which will be therapeutic for at least about 90% of the adult population in the United States. In this regard, an analysis by the USDA of the diet of a multitude of male volunteers over 50 years of age established that the average daily consumption of DHA that captured 90% of these adults (90^{th} percentile) was up to about 130 mg/day. Given that males tend to eat more than females, one can reasonably conclude that this level of DHA consumption captured more than 90% of all patients including females. By extrapolation one can capture many of the remaining 10% of patients with higher daily DHA intakes to be treated using an initial fixed daily dose of about 250 mg, or about 500 mg, or about 1,000 mg per day of D-DHA. The higher the initial fixed doses, the greater the number of patients who will be captured.

In some embodiments, the oxidative neurodegenerative disease is Amyotrophic Lateral Sclerosis (ALS), Jacobson Syndrome, spinal muscular atrophy, multiple system atrophy, Alzheimer's Disease (AD) and mild cognitive impairment, Huntington's Disease, infantile neuroxonal disease (INAD), Parkinson's Disease, or Progressive supranuclear palsy (PSP).

In some embodiments, the oxidative retinal disease is wet and dry age-related macular degeneration (including geographic atrophy associated therewith), retinal degeneration, cataracts, retinitis pigmentosa, diabetic retinopathy, glaucoma, or Stargardt Disease.

In some embodiments, this disclosure provides for pharmaceutical compositions comprising from an amount of D-DHA sufficient to provide at least about a 1:4 ratio of D-DHA to DHA in a patient after steady state concentration of active D-DHA is reached in blood. In some embodiments, such pharmaceutical compositions comprise about fixed amounts of 50 mg of D-DHA, or about 100 mg of D-DHA, or about 250 mg of D-DHA, or about 500 mg of D-DHA, or about 1,000 mg of DHA and optionally a pharmaceutically acceptable carrier.

It is noted that the initial dosing of D-DHA is done based on published statistical data in the absence of any assays conducted on the patient but rather might include a completed dietary questionnaire of what foods are consumed and how often. Such a questionnaire would include inquiries as to the amount and types of seafood consumed. Upon the review of the questionnaire, the clinician can either advise to reduce consumption of DHA-containing supplements or oily fish or select a dose of D-DHA that best corresponds to the patient's actual diet. For example, as shown in the examples, patients who are administered 250 mg D-DHA per day and who consume on average about 130 mg per day of DHA, will have a ratio of D-DHA to DHA of about 1.92 to 1 when steady state concentrations are reached for each of the plasma, red blood cells and large organs such as liver and skeletal muscle tissue. As the average amount of DHA consumed by the patient is reduced from 130 mg/day, the ratio is increased in favor of D-DHA. Likewise, for patients assigned higher doses of D-DHA while consuming 130 mg/day of DHA, higher ratios of D-DHA to DHA are obtained.

The exact dose initially assigned to the patient by the attending clinician may be predicated on the target ratio of active D-DHA to DHA that is appropriate for that patient based on the age, weight, sex, particular oxidative disease being treated. Afterwards, when the average daily amount of DHA consumed by the patient has been determined, the clinician can adjust the dose of D-DHA to achieve a target ratio all of which are within the skill of the attending clinician. In general, it is considered that a dose of either 250 mg per day of D-DHA, or 500 mg/day of D-DHA or a dose of 1,000 mg/day of D-DHA will be appropriate to therapeutically treat greater than 95% of all patients when dosed at 250 mg/day and greater than 99% of all patients treated when dosed at 1,000 mg/day.

Accordingly, in another aspect, there is provided a pharmaceutical composition comprising a dose of at least about 250 mg of D-DHA (or about 250 mg of D-DHA) in one or more pharmaceutically acceptable units and optionally in the presence of a pharmaceutically acceptable carrier.

In some embodiments, there is provided a pharmaceutical composition comprising unit dose of at least about 500 mg of D-DHA (or about 500 mg of D-DHA) in one or more pharmaceutically acceptable units and optionally in the presence of a pharmaceutically acceptable carrier.

In some embodiments, there is provided a pharmaceutical composition comprising of at least about 1,000 mg of D-DHA (or about 1,000 mg of D-DHA) in one or more pharmaceutically acceptable units and optionally in the presence of a pharmaceutically acceptable carrier.

In some embodiments, the D-DHA administered is an ester of docosahexaenoic acid. In another embodiment, the ester is a C₁-C₆ alkyl ester and preferably the ethyl ester.

In some embodiments, once the clinician knows what the average daily consumption of DHA is coupled with the amount of D-DHA administered, the clinician can then utilize standardized curves [see FIGS. 2, 3, 4, 5, and 6 as examples] to ascertain what the concentration of active D-DHA should be in the patient at different times after start of therapy. Such curves allow the clinician to monitor and evaluate the overall accumulation of active D-DHA in the target tissues or cells for a given patient. If a patient fails to achieve a predicted concentration of active D-DHA from a given date after start of therapy in either the plasma or the red blood cells based on standardized curves for the dose of D-DHA used, the clinician can decide to provide a bolus of D-DHA to the patient, to limit or further limit the amount of DHA consumed by the patient to an average of no more than 130 mg/day and/or increase the dose of D-DHA.

Still further, the steady state concentration of active D-DHA in either plasma or red blood cells correlates well with the steady-state concentration in the body. For example, steady state concentrations of active D-DHA in neurons and in the retina are reached about 4 to about 7 months after start of therapy. In addition, when a targeted steady state concentration in blood is achieved for a given dose of D-DHA, it evidences that continued administration of D-DHA to treated patients will later achieve the desired concentration of the active D-DHA in their targeted tissues/organs relative to the dose of D-DHA administered. In addition, it has been found that when steady state concentrations are maintained in the blood for the periods of time described above, the clinician can confirm that a steady concentration will also correlate to a therapeutic concentration in the retina and the neurons. As also shown in the examples, steady state concentration of active D-DHA (ratio of D-DHA to DHA) are the same in all tissues.

Accordingly, in another aspect, there is provided a method for monitoring a patient for uptake of active D-DHA wherein the method comprises:
administering to the patient a fixed dose of D-DHA;
obtaining one or more blood or blood component samples from the patient at a set point after the start of therapy;
assessing the ratio of active D-DHA to DHA in the sample relative to the total amount of DHA;
comparing the assessed amount of active D-DHA against a standard concentration curve wherein the curve is based on a specific dose of D-DHA, the average daily amount of DHA consumed by the patient, the blood or blood component being assessed, and the length of time from start of therapy;
determining if the patient is achieving targeted active D-DHA to DHA ratios based on the curve; and
increasing the dosing of D-DHA as necessary to reach or exceed the targeted active D-DHA to DHA ratio and/or restricting the amount of DHA consumed by the patient.

In some embodiments, any substantial deviation in the ratio of active D-DHA to DHA from a patient's previous results will require verification as to what factors may have produced such a result. For example, the clinician can inquire as to patient's diet immediately prior to the blood test and, as appropriate, instructing the patient to refrain from fats such as oily seafood or fish oil for at least 7 days prior to the follow-on blood test. The clinician can also inquire as to whether the patient has ceased therapy for an extended period of time well in excess of the accountable non-compliance days provided herein.

In some embodiments, the blood component being assessed is plasma.

In some embodiments, the blood component being assessed is red blood cells.

In some embodiments, the length of time between start of therapy and initial blood testing is from about 14 to about 120 days. In some embodiments, the length of time between start of therapy and initial blood testing is at least about 14 days. In another embodiment, the length of time between start of therapy and initial blood testing is at least about 30 days or at least about 90 days.

In some embodiments, the method for monitoring a patient for uptake of active D-DHA employs a dose of D-DHA of about 250 mg/day, or about 500 mg/day or about 1,000 mg/day of D-DHA.

### BRIEF DESCRIPTION OF THE DRAWINGS

FIG. 1 shows the detected increase in concentration of D-DHA using three different markers (plasma, red blood cells and the retina) in a cohort of mice on a customized rodent diet containing 0.5% w/w D-DHA at four different time points (8, 19, 38, and 77 days after first D-DHA exposure).

FIGS. 2, 3, 4, 5, and 6 illustrate predictive standardized curves modeled from the data of FIG. 1 showing the increase in concentration of D-DHA for five different dosing regimens and using different markers (plasma, red blood cells and the retina) over a time course from start of therapy to reaching steady state in patients with a mean dietary intake of about 130 mg DHA per day.

FIG. 7 illustrates variations in D-DHA concentration as a function of variable dietary DHA intakes at state ratios between about 1.4:1 and about 3.6:1 D-DHA to DHA modeled from the data of FIG. 1, which represent the differences between the mean (70 mg/day), the 90^{th} percentile (130 mg/day), and the 95^{th} percentile (180 mg/day) of dietary DHA intakes in eldery males in the US.

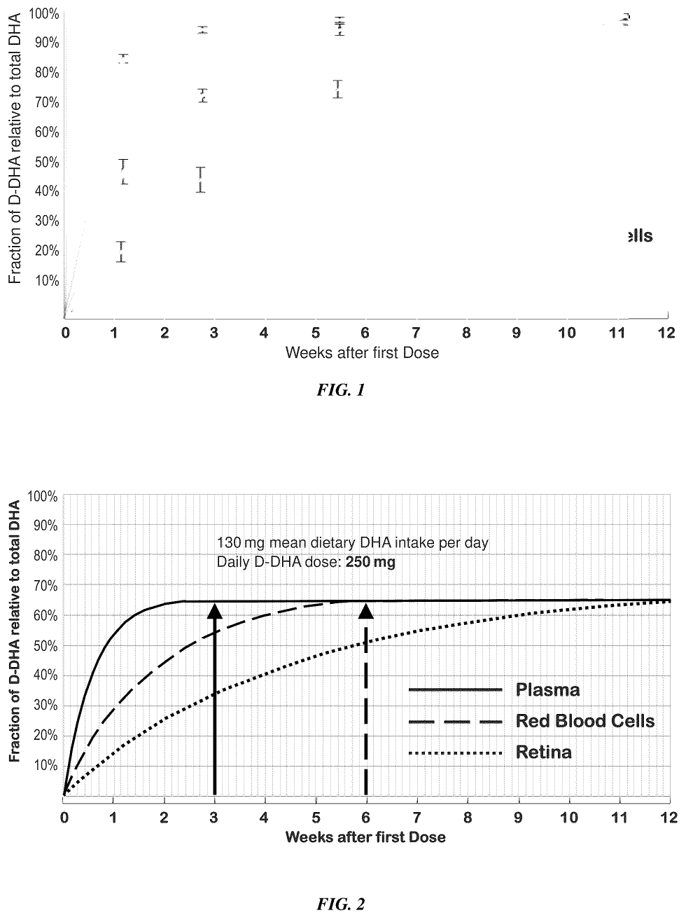

### DETAILED DESCRIPTION

Disclosed are pharmaceutical compositions comprising D-DHA or an ester or a pharmaceutically acceptable salt thereof (“D-DHA”) as well as methods related thereto. Before describing these compositions and methods in more detail, the following terms are defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−5%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein and unless the context dictates otherwise, the term “an ester thereof” refers to a C₁-C₁₀ alkyl esters, glycerol esters (as defined herein and including monoglycerides, diglycerides and triglycerides), sucrose esters, phosphate esters, and the like. The particular ester group employed is not critical provided that the ester is pharmaceutically acceptable (non-toxic and biocompatible). In some embodiments, the ester is a C₁-C₆ alkyl ester that is preferably an ethyl ester.

As used herein, the term “alkyl” refers to straight, branched and cyclic alkyl groups such as methyl, ethyl, n-propyl, isopropyl, cyclopropyl, n-butyl, isobutyl, sec-butyl, t-butyl, cyclobutyl, 2-methylcyclopropyl, n-pentyl, cyclopentyl, n-hexyl, cyclohexyl, and the like.

As used herein, the term “steady state ratio” refers to the ratio of active D-DHA to DHA in vivo that is maintained at between about 90% and 110% of its maximal value based on the dose of D-DHA administered and the average daily consumption of DHA by the patient. As noted above, a patient consuming on average 130 mg/day of DHA and having administered about 250 mg/day of D-DHA will have a D-DHA to DHA ratio of about 1.92 to 1. So, in this case, a steady state concentration of D-DHA to DHA will be from about 1.73:1 to about 2.11:1.

Since the average daily consumption rate of DHA varies day to day, the steady state ratio will fluctuate somewhat based on the amount of DHA consumed. In addition, the amount of D-DHA administered is not likely to be at the same time each day and, in fact, the patient may be medication non-compliant on a given day. FIG. 7 is intended to show the variation in the steady state ratio of D-DHA to DHA when D-DHA is administered at 250 mg per day to a hypothetical patient on average 130 mg DHA per day while having a minimum consumption of DHA of about 70 mg/day and a maximum of about 180 mg/day. In FIG. 7, the slight oscillation up in the ratio of D-DHA to DHA at steady state is indicative of less daily DHA being consumed for a period of several weeks and the slight oscillation down in the ratio of D-DHA to DHA is indicative of more daily DHA being consumed for a period of several weeks.

As used herein, the terms “deuterated DHA”, “D-DHA” or “deuterated docosahexaenoic acid or ester thereof” refers to a docosahexaenoic acid as well as esters thereof having deuteration as described below. In vivo, esters are first hydrolyzed to provide for the corresponding acid (“active D-DHA”) thereof and then incorporated into structural features such as glycerol esters. Prior to describing deuteration, the structure of docosahexaenoic acid and specific sites therein are provided in formula (A) below:

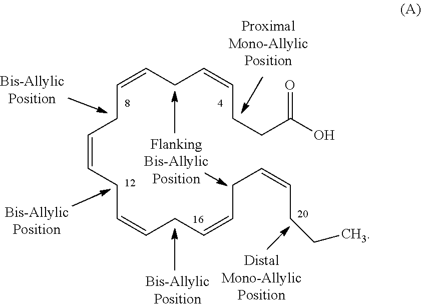 [SMILES: CCC/C=C\C/C=C\C/C=C\C/C=C\C/C=C\C/C=C\CCC(=O)O]

As to deuteration, such is described as an average based on a population of such DHA compounds comprising a total deuteration of at least about 80 percent at the bis-allylic sites (replacing 80% of the hydrogen atoms at these sites with deuterium atoms) and about 30% or less of total deuteration at the two mono-allylic sites (about 30% or less of the 4 hydrogen atoms at the two mono-allylic sites have been replaced with deuterium).

In apreferred embodiment, replacement of hydrogen with deuterium at the bis-allylic sites is from about 92 to about 96 percent wherein the total deuteration is present as follows:
a) from about 87 percent to about 92 percent CD₂ moieties at the bis-allylic sites;
b) from more than about 6 about to 12 percent CHD moieties at the bis-allyic sites; and
c) about 2 percent or less of CH₂ moieties at the bis-allylic sites, provided that the aggregate number of hydrogen and deuterium at the bis-allylic positions equals 10.

In another preferred embodiment, the bis-allylic sites of D-DHA have a total deuteration of from about 93 to about 96 percent at the bis-allylic sites wherein the total deuteration is present as follows:
a) from about 87 to about 92 percent CD₂ moieties at the bis-allylic sites;
b) from about 6.5 to about 12 percent CHD moieties at the bis-allylic sites; and
c) about 1.5 percent or less of CH₂ moieties at the bis-allylic sites, provided that the aggregated number of deuterium and hydrogen atoms at the bis-allylic positions equals 10.

In yet another preferred embodiment, the bis-allylic sites of D-DHA have a total deuteration of from about 92 to about 95 percent at the bis-allylic sites wherein the total deuteration is present as follows:
a) about 88 to 92 percent CD₂ moieties at the bis-allylic sites;
b) about 6.5 to 12 percent CHD moieties at the bis-allyic sites;
c) about 1.5 percent or less of CH₂ moieties at the bis-allylic sites; and
d) on average no more than an aggregate of 25% total deuteration at both mono-allylic sites

provided that the total number of hydrogen and deuterium at the bis-allylic positions equals 10.

The extent of deuteration at the two mono-allylic differs due to the steric hindrance imparted by the carboxyl or carboxyl ester. In some embodiments, the extent of deuteration at the proximal mono-allylic site is from about 0.5% to about 5%. In another embodiment, the extent of deuteration at the proximal mono-allylic site is from about 1% to about 5%. Stated differently, on average, only about 0.5% to about 5% or 1% to about 5% of the hydrogen atoms found at the proximal mono-allylic site of a composition comprising a population of D-DHA have been replaced by deuterium.

In some embodiments, the level of deuteration at the distal mono-allylic site is from about 10% to about 20%. In some embodiments, the level of deuteration at the distal mono-allylic sites is from about 12% to about 18%. Stated differently, on average, only about from 10% to about 20% or from 12% to about 18% of the hydrogen atoms found at the distal mono-allylic site of a composition comprising a plurality of D-DHA have been replaced by deuterium.

In some embodiments, the deuterated docosahexanoic acid or ester thereof comprises a population of a compound of formula (I):

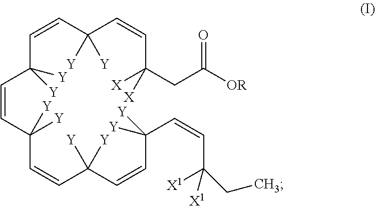

where R is hydrogen, a C₁-C₁₀ alkyl or a pharmaceutically acceptable salt;

each X is independently hydrogen or deuterium wherein the aggregate amount of the amount of deuterium defined by both X groups is such that, on average, the total amount of deuteration at the carbon atom is less than about 5%;

each X¹ is independently hydrogen or deuterium wherein the aggregate amount of the amount of deuterium defined by both X¹ is such that, on average, less than about 25% of the X¹ groups are deuterium and the remainder are hydrogen;

each Y is independently hydrogen or deuterium wherein the specific value for each Y is selected such that on average:
a) from about 87 to about 92 percent of the Y groups on each carbon atom are deuterium;
b) from more than about 5 about to 12 percent of the Y groups on each carbon atom are substituted with a single hydrogen and a single deuterium; and
c) less than about 2 percent of the Y groups of each carbon atom are substituted with two hydrogen atoms;

provided that the sum of all Y groups equal 10.

In some embodiments, the composition comprising the compound of formula (I) comprises less than 1.5 percent of the carbon atoms at the bis-allylic sites being substituted with two hydrogen atoms.

In some embodiments, the compositions described herein do not replace hydrogen with deuterium other than at the mono-allylic and bis-allylic sites. As such, the level of deuterium found in the remaining sites in DHA is at its natural abundance.

Examplary deuterated DHA compositions described herein are provided in Table 1 below and referencing formula (I) above:

TABLE 1

| | | Average percent | Average percent |
|---|---|---|---|
| | Average percent | deuterium at both | deuterium at both |
| | deuterium at the | X mono- | X¹ mono- |
| Example | bis-allylic sites | allylic sites | allylic sites |
| 1 | at least | from about 15 | from about 1 |
| | about 80% | to about 35% | to about 15% |
| 2 | at least | from about 15 | from about 2 |
| | about 85% | to about 30% | to about 15% |
| 3 | at least | from about 15 | from about 3 |
| | about 85% | to about 25% | to about 15% |
| 4 | at least | from about 10 | from about 2 |
| | about 90% | to about 25% | to about 12% |
| 5 | at least | from about 10 | from about 2 |
| | about 90% | to about 20% | to about 10% |
| 6 | at least | from about 5 | from about 2 |
| | about 95% | to about 20% | to about 10% |
| 7 | at least | from about 5 | from about 2 |
| | about 95% | to about 15% | to about 10% |

In one preferred embodiment, the aggregate of both X groups contains from about 5% to about 30% of deuterium including all subranges between these two numbers whereas the aggregate of both X¹ groups contains from about 1% to about 10% of deuterium including all subranges between these two numbers.

In some embodiments, the deuterated DHA is provided as a pharmaceutical composition comprising an effective amount of deuterated DHA or an ester or a pharmaceutically acceptable salt thereof as described herein and optionally a pharmaceutically acceptable carrier.

When describing the population ex vivo, the terms “D-DHA” or drug” refer to deuterated docosahexaenoic acid, or an ester thereof or a pharmaceutically acceptable salt thereof. When describing the population in vivo, the ester is hydrolyzed in the gastro-intestinal tract and, in the environment of the retina, docosahexaenoic acid is incorporated into a glycerol ester such as a phospholipid, including cardiolipin, plasmalogen and those of the formula (II):

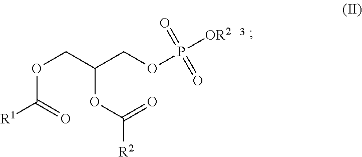

where R¹ is a fatty acid residue or the residue of docosahexaenoic acid, R² is the residue of docosahexaenoic acid, and R³ is choline, ethanolamine, serine, inositol or hydrogen, a mono- or divalent salt. Unlike fatty acids found elsewhere in the body, the retina can comprise residues of deuterated docosahexaenoic acids at both R¹ and R². Accordingly, this invention provides for phospholipids of formula (II) where R¹ is selected from a residue of a saturated fatty acid or the residue of docosahexaenoic acid and R² is the residue of docosahexaenoic acid. As to the terms “residue of a fatty acid” or “residue of docosahexaenoic acid”, each of these refers to the ester bond formed between a carboxyl group and a hydroxyl group of glycerol coupled with the elimination of water.

In a preferred embodiment, deuteration at other sites of docosahexaenoic acid or an ester thereof is unaffected and, hence, the level of deuteration at sites other than the bis-allylic and mono-allylic sites is at the natural abundance.

The term “naturally occurring docosahexaenoic acid” refers to any and all sources of DHA where the abundance of deuterium is based on its natural abundance.

As used herein, the term “phospholipid” refers to any and all phospholipids that are components of the cell membrane. Included within this term are phosphatidylcholine, phosphatidylethanolamine, phosphatidylserine, and sphingomyelin. In the motor neurons, the cell membrane is enriched in phospholipids comprising arachidonic acid.

The term “bis-allylic site” refers to the methylene group (CH₂) separating two double bonds.

The term mono-allylic site” refers to the methylene group have an adjacent neighboring double bond on one side and a further methylene group on the opposite side.

The term “oxidized PUFA products” refer to any oxidized form of a polyunsaturated fatty acid as well as any and all metabolites formed from the oxidized PUFA including reactive aldehydes, ketones, alcohols, carboxyl derivatives which are toxic to the cell where found in a phospholipid, a lipid bilayer, or as an enzyme substrate.

As used herein, the term “etiology of a disease” refers to the underlying cause or causes of the disease. The term “pathology of a disease” refers to the development, structural/functional changes, and natural history associated with that disease after the disease is cause. Included in the pathology of the disease is the reduction in cellular functionality.

The term “therapeutic concentration” means a concentration of active D-DHA that reduces the rate of an oxidative disease mediated, at least in part, by lipid peroxidation. As set forth in the examples below, such a concentration is predicated on replacing at least about 20 percent of the DHA in target tissue or cells with D-DHA as described herein and preferably at least about 33 percent, preferably at least about 50 percent, more preferably at least about 75%, and most preferably at least about 80 percent. To achieve this level of replacement level, dosing of D-DHA over a period of time (weeks to several months) is necessary as active D-DHA is slowly exchanged in vivo.

In some embodiments, the targeted tissue is the retina and, in particular, the rods and cones in the retina, especially in the treatment of oxidative retinal diseases. Such diseases include, but are not limited to but are not limited to, wet and dry age-related macular degeneration (including geographic atrophy associated therewith), retinitis pigmentosa, diabetic retinopathy, cataracts, and Stargardt Disease.

In some embodiments, neurons are targeted including, but not limited to, motor neurons and memory neurons where lipid peroxidation leads to oxidative neuronal diseases. Such neuronal oxidative diseases include, but are not limited to, Amyotrophic Lateral Sclerosis (ALS), Jacobson Syndrome, spinal muscular atrophy, multiple system atrophy, atherosclerotic vascular disease, Alzheimer's Disease (AD) and mild cognitive impairment, Huntington's Disease, infantile neuroxonal disease (INAD), Parkinson's Disease, to name a few.

As used herein, the terms “blood” or “blood or component thereof” refers to either red blood cells or plasma.

As used herein, the term “non-compliance” means that a patient is medication non-compliant at a rate that does not permit achieving and/or maintaining a steady state ratio of D-DHA to DHA of at least about 1:4 during treatment.

As used herein, the term “accountable days of patient non-compliance” or “accountable days of medication non-compliance” means that the dosing regimen of D-DHA is sufficient to account for or tolerate a number of days of patient non-compliance per month while maintaining a ratio of D-DHA to DHA of at least about 1:4 for that patient during therapy. The extent of accountable days of non-compliance per month is dependent on the dose employed and the amount of DHA consumed. Since higher doses of D-DHA provide for higher ratios of active D-DHA to DHA at steady state in the liver and skeletal muscle tissue independent of the average daily consumption ratio of DHA, the clinician may choose to dose a patient with about 250 mg/day, or 500 mg/day, or 1,000 mg/day of D-DHA in order to increase then number of accountable days of medication non-compliance for at least about 4 days per month at 250 mg/day and up to at least about 7 days per month when dosed at 500 mg/days of D-DHA or an ester or a pharmaceutically acceptable salt thereof both starting with patients having no more than about 130 mg/day intake of DHA.

For patients having an average intake of DHA of about 260 mg/day or less due to their consumption of on average one oily seafood meal per week on average, the dose of D-DHA or an ester or a pharmaceutically acceptable salt thereof is increased to about 500 mg/day with up to at least about 7 days per month of accountable medication non-compliance days. At a dose of D-DHA of about 1,000 mg/day, the number of accountable medication non-compliance days per month is at tleast about 10.

In a preferred embodiment, availability of accountable days of medication non-compliance is predicated on first achieving a therapeutic steady state concentration of D-DHA in large DHA storing organs such as in skeletal muscle which can be at least as long as 2.5 months after start of therapy. As such, the accountable days of medication non-compliance preferably occurs afterwards and is associated with long-term therapy where medication non-compliance is particularly acute.

The finding that one can account for medication non-compliance days in the compositions and methods described herein is both unexpected and medically important and correlates to a higher rate of efficacy for a drug. Stated differently, the medication non-compliance is an ongoing medical issue especially for chronic diseases requiring treatment for the remaining portion of the patient's life. In general, medication compliance is viewed from both short-term mediation compliance (about 2.5 months or less) and long-term or chronic medication compliance (greater than about 2.5 months).

In general, short-term medication compliance has been estimated to range from 70 to 80 percent with higher percentages provided for shorter medication compliance. In contrast thereto, long-term medication compliance range has been estimated to range from about 40 to 50 percent. See, e.g., ncbi.nlm.nih.gov/pmc/articles/PMC2503662/which is incorporated herein by reference in its entirety. So, the initial stage of patient treatment is reached when a steady state concentration of active D-DHA is provided in major tissues such as the skeletal muscle tissue which occurs about 11 weeks (2.5 months) after start of therapy. See, e.g., Table 2 below.

TABLE 2

| | Accretion | | | | | Time to | Elimination | | | | | Terminal |
|---|---|---|---|---|---|---|---|---|---|---|---|---|
| | Half-Life | Days to Increase D-DHA level | | | | Steady | Half-Life | Days to Decrease D-DHA level | | | | Half-life |
| Tissue | in Days | (Substitution Rate) by: | | | | State | in Days | (Substitution Rate) by: | | | | in Days |
| (ranked by t_{1/2a}) | (t_{1/2a})* | 10% | 20* | 30% | 40% | (5 × t_{1/2a}) | (t_{1/2e})* | 10% | 20% | 30% | 40% | (5 × t_{1/2e}) |
| Plasma | 2.70 | 0.41 | 0.87 | 1.39 | 1.99 | 13.5 | 23.67 | 3.60 | 7.62 | 12.18 | 17.44 | 118.4 |
| Liver | 2.85 | 0.43 | 0.92 | 1.47 | 2.10 | 14.3 | 14.21 | 2.16 | 4.57 | 7.31 | 10.47 | 71.1 |
| RBC | 8.75 | 1.33 | 2.82 | 4.50 | 6.45 | 43.8 | 36.52 | 5.55 | 11.76 | 18.79 | 26.91 | 182.6 |
| Skeletal Muscle | 14.76 | 2.24 | 4.75 | 7.60 | 10.88 | 73.8 | 15.39 | 2.34 | 4.95 | 7.92 | 11.34 | 77.0 |
| Retina | 23.42 | 3.56 | 7.54 | 12.05 | 17.26 | 117.1 | 22.78 | 3.46 | 7.33 | 11.72 | 16.79 | 113.9 |
| Optic Nerve | 26.26 | 3.99 | 8.45 | 13.51 | 19.35 | 131.3 | 22.37 | 3.40 | 7.20 | 11.51 | 16.49 | 111.9 |
| Cerebellum | 29.00 | 4.41 | 9.34 | 14.92 | 21.37 | 145.0 | 29.33 | 4.46 | 9.44 | 15.09 | 21.62 | 146.7 |
| Cortex | 41.40 | 6.29 | 13.33 | 21.30 | 30.51 | 207.0 | 37.18 | 5.65 | 11.97 | 19.13 | 27.40 | 185.9 |
| Hippocampus | 44.26 | 6.73 | 14.25 | 22.77 | 32.62 | 221.3 | 35.84 | 5.45 | 11.54 | 18.44 | 26.41 | 179.2 |

During this initial medication compliance period, medication non-compliance is less of a problem. This is because when patients are informed that they have a devastating disease such macular degeneration, ALS, and the like, they are significantly more likely to maintain medication compliance during the first 2.5 months from the start of therapy. However, for life-long chronic therapy, there is a significant drop off in compliance when transitioning to long-term medication. This is particularly the case where disease progression does not materially change on a day-to-day basis. It is during this stage of treatment that the benefits of accountable days of medication non-compliance are particularly important.

Without being limited to any theory, the ability to achieve accountable days of medication non-compliance is because D-DHA exhibits a large therapeutic window that allows for dosing up to about 1,250 mg/day and is stored for long periods of time in the body once steady state concentration is reached in the skeletal muscle tissue. Specifically, as shown in Table 2 above, the elimination half-life for active D-DHA in the skeletal muscle tissue is about 15.4 days, about 14.2 days for the liver, and about 23.7 days for plasma. This means that when steady state concentrations of active D-DHA are achieved in tissues with significant DHA storage capacity such as liver and the skeletal muscle tissue, these tissues act as a significant active D-DHA drug depot.

Again, without being limited to any theory, for patients whose target in vivo ratio of active D-DHA to DHA is 1:4 and that patient is consuming on average about 200mg/day of DHA and is being treated with 50 mg/day of D-DHA, that patient should reach a ratio of active D-DHA to DHA in the skeletal muscle tissue at about 75 days of about 0.2:1. Given that such a ratio is the minimal ratio for therapeutic results, there will be no days of accountable medication non-compliance

Example 7 illustrates a theoretical example of just how the ratio of active D-DHA to DHA can vary based on the amount of DHA consumed per day

As used herein, the term “patient” refers to a human patient or a cohort of human patients suffering from a neurodegenerative disease treatable by administration of deuterated DHA. The term “subject” refers to a mammalian subject.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

The term “pharmaceutical unit” means that the drug is delivered daily in one or more pills, tablets, capsules, liquid formulations, etc. The particular form of the pharmaceutical unit is not critical and is within the discretion of the clinician. In one preferred embodiment, the drug is encapsulated within a capsule as a liquid without the addition of any pharmaceutical excipient. In another preferred embodiment, the drug is encapsulated within a capsule as a liquid using a pharmaceutical excipient such as biologically acceptable oil (e.g., olive oil, sunflower oil, coconut oil, etc.).

The term “oily fish” means fish that contain at least 500 mg of DHA in a three (3) ounce serving. Such fish include, but not limited to, salmon, herring, tuna, trout, and the like.

### Compound Synthesis

Deuterated DHA compositions as described herein are obtained as per U.S. Pat. No. 10,577,304 which is incorporated herein by reference in its entirety.

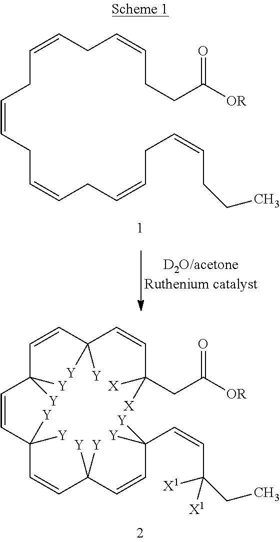

where R, X, X¹, and Y are as defined above.

As to Scheme 1, the reaction can be conducted using docosahexaenoic acid ethyl ester (or any other suitable ester), compound 1, a stoichiometric excess of deuterium oxide in a suitable inert solvent in the presence of the ruthenium catalyst as described in U.S. Pat. No. 10,577,304 which is incorporated herein by reference in its entirety.

As shown in Example 1 herein, the total amount of deuteration at the bis-allylic sites ranges from about 92 to about 97 percent. Stated differently, after deuteration the 10 hydrogen atoms at the bis-allylic sites have been replaced with, on average, between about 9.2 and about 9.7 deuterium atoms leaving only about 0.3 to 0.8 hydrogen atoms. Moreover, high field NMR establishes that on average from about 87 to about 92 percent of the carbon atoms at the bis-allylic sites have two deuterium atoms and from more than about 5 about to 12 percent of the carbon atoms at the bis-allyic sites have a single hydrogen and a single deuterium substitution with the residual being CH₂ moieties.

Given the above, it has been determined that even though complete deuteration of the bis-allylic sites has not been achieved, the presence of CHD groups at these sites impart greater stability against lipid peroxidation than the CH₂ groups. By limiting the reaction conditions such that, on average, no more than about 2 percent of the carbon atoms at the bis-allylic sites are CH₂ groups, the resulting composition still provides for excellent control of against LPO in vivo.

### Pathology

The resulting pathology of each of the oxidative diseases (including retinal oxidative diseases and neuronal oxidative diseases) is different from the underlying etiology of other oxidative disease but proceed via a common pathology. That is to say that whatever divergent conditions trigger each of these oxidative diseases (the etiology), once triggered the pathology of these diseases includes the accumulation of oxidized DHA products. By limiting the oxidative damage, the pathology of the disease is addressed and the detrimental impact on the patient is mitigated. In the case of AMD as an example, animal studies evidence that the degradation of eyesight induced by retinal lipid peroxidation arising from ferroptosis is significantly limited by treating the animal with deuterated DHA as compared to the untreated animals.

Without being limited to any theory and in the case of oxidative disease, the incorporation of deuterated DHA into the outer segments of rods and cones of the retina and surrounding retinal tissues limit the degree of oxidation by reactive oxygen species. This, in turn, protects the cells in the retina from damage and destruction typical of retinal oxidative diseases (e.g., AMD).

Again, without being limited to any theory and in the case of neuronal diseases, the incorporation of D-DHA into the neuron's cellular membranes limits the degree of oxidation by reactive oxygen species thereby limiting cellular dysfunction or cellular death.

### Dosing

The methods described herein entail the periodic dosing of D-DHA as described herein to both achieve a therapeutic concentration and to maintain such a concentration in vivo. The dosing employed herein accounts for the variability of individual patients' diets with regard to the daily dosing of D-DHA. In addition, the gradual increase in the in vivo concentration of D-DHA and its relatively long half-life allows for the clinician to account for days of non-compliance with the dosing regimen while the results for that patient are still considered to be probative for efficacy of the drug.

Accordingly, a patient who intentionally or inadvertently misses a daily dose of the drug is still compliant with the overall dosing protocol which is quite dissimilar to conventional drugs.

The dosing regimen employs a daily or unit dose of from about 100 mg/day to about 1,250 mg/day without regard to the patient's BMI, severity of the disease condition, the otherwise overall health of the patient or initially the consumption level of DHA as noted above.

The diagnosis and progression of the oxidative ocular disease is evaluated by any one of a number of conventional diagnostic tools well known in the art. See, e.g., verywellhealth.com/how-macular-degeneration-is-diagnosed-4160590. In some embodiments, the rate of reduction in a patient's disease progression is evaluated by comparing the ocular tests results subsequent to start of therapy to those obtained at the time of the original diagnosis/start of therapy or to the test results from any prior evaluation. The data suggest that the rate of disease progression in an individual patient will be reduced by at least about 20%, or at least about 30%, or at least about 40%, or at least about 50% or more when the dosing methods described herein are employed. The amount of reduction may be any value or subrange within the recited ranges, including endpoints. In general, the comparison is between the known rate of disease progression and that experienced by the patient and is made at any time from 1 to 24 months, such as about 6, or 12, or 18, or 24 months after initiation of therapy and then periodically thereafter (e.g., every 6 months). In some embodiments, the known rate of disease progression can be based on the rate of geographic atrophy progression in a cohort of patients treated with placebo over the same period of time.

In another embodiment, the efficacy of the treatment protocol can be evaluated by comparing the extent of geographic atrophy progression in a treated population or individual against a placebo population. In such a comparison, efficacy is established by a statistically significant reduction in geographic atrophy progression in the treated population as compared to the placebo population. Preferably, the degree of reduction is at least by at least about 20%, or at least about 25%, or at least about 30%, or at least about 50% or more when the dosing methods described herein are employed.

The methods described herein are also based, in part, on the discovery that when the lipid membrane of the cells such as neurons is stabilized against LPO, there is a substantial reduction in the progression of the oxidative neuronal disease. Without being limited by theory, it is believed this is because replacement of hydrogen atoms with deuterium atoms at the deuterated docosahexaenoic acid bis-allylic sites renders these carbon-deuterium bonds significantly more stable to ROS than the carbon-hydrogen atoms. As above, this stability manifests itself in reducing the cascade of lipid auto-oxidation and, hence, limiting the rate of disease progression.

### Combinations

The therapy provided herein can be combined with any other treatments used with oxidative retinal diseases provided that such treatment does not interfere with the therapy described herein. In the case of macular degeneration, drugs such as bevacizumab, ranibizumab. aflibercept, and brolucizumab have all been prescribed to attenuate disease progression and can be used in combination with the therapy described herein.

In another embodiment, a combination therapy can employ a drug that operates via an orthogonal mechanism of action relative to the methods described herein. Suitable drugs for use in combination include, but not limited toflavonoids, resveratrols, carotenoids, cyanines and antioxidants such as edaravone, idebenone, mitoquinone, mitoquinol, vitamin C, or vitamin E, riluzole which preferentially blocks TTX-sensitive sodium channels, conventional pain relief mediations, and the like.

### Pharmaceutical Compositions

The specific dosing of deuterated docosahexaenoic acid or an ester thereof described herein is accomplished by any number of the accepted modes of administration. As noted above, the actual amount of the drug used in a daily or periodic dose per the methods of this invention, i.e., the active ingredient, is described in detail above. The drug can be administered at least once a day, preferably once or twice or three or more times a day.

This invention is not limited to any particular composition or pharmaceutical carrier, as such may vary. In general, compounds of this invention will be administered as pharmaceutical compositions by any of a number of known routes of administration. However, orally delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, the docosahexaenoic acid or an ester thereof is administered in a gel capsule as a neat oil.

Pharmaceutical dosage forms of a compound of this invention may be manufactured by any of the methods well-known in the art, such as, by conventional mixing, tableting, encapsulating, and the like. The compositions of this invention can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions of this invention may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 100 weight percent of the drug based on the total formulation outside of the weight of the capsule carrier with the balance being one or more suitable pharmaceutical excipients. Preferably, the compound is present at a level of about 50 to 99 weight percent.

In a preferred embodiment, the drug is encapsulated inside a capsule without the need for any pharmaceutical excipients such as stabilizers, antioxidants, colorants, etc.

In a preferred embodiment, D-DHA administered to patients is a docosahexaenoic acid ester such as a C1-C6 alkyl ester and preferably the ethyl ester.

### Methods

In some embodiments, the methods described herein comprise the administration of D-DHA to a patient suffering from an oxidative retinal or neuronal disease.

The drug is delivered to the patient at a dose prescribed by attending clinician. Typically, such a dose is from about 100 to about 1,250 milligrams/day. The accumulation of active D-DHA in the body can be monitored by, for example, blood tests to ensure that the patient is accumulating active D-DHA consistent with achieving a therapeutic result. If the blood tests evidence insufficient levels of active D-DHA, the clinician can determine if the dietary intake of DHA should be adjusted, dosing should be increased, or assessing whether the patient has terminated administration of the drug.

In a preferred embodiment, D-DHA is administered to the patient in sufficient amounts to generate a steady state concentration of active D-DHA in blood of at least about 25% (a 1:3 ratio of D-DHA to DHA), or at least about 33% (a 1:2 ratio), or at least about 50% (a 1:1 ratio), or at least about 66% (a 2:1 ratio) or at least about 80%, (a 4:1 ratio) based on the total amount of DHA, including active D-DHA, found therein. In an embodiment, the percentage of active D-DHA compared to total DHA in a patient (e.g., in the red blood cells, plasma, and/or retinal cells) may be between about 25% and about 80%, between about 33% and about 70%, or between about 50% and about 66%.

### Testing Protocols

Once administered, the attending clinician needs to monitor the rate of absorption of active D-DHA into the target tissue or cells. As physical access to the retina or to neurons is not feasible in live animals, the methods described in the examples illustrate that either blood plasma or red blood cells (RBCs) can be used as a proxy for assessing whether absorption is proceeding properly in the retina. This is because the plasma and RBCs both reach steady state concentrations which occur at different times after the start of therapy but nevertheless allow for the clinician to conclude that the patient will reach steady state concentrations in the target tissue or cells at times subsequent thereto as noted above in Table 2.

Testing blood plasma or RBCs for the presence of individual components contained therein is well established in the art. However, the art has been unable to establish a correlation between the presence and the amount of DHA in blood and the daily dietary intake of DHA. The examples below now evidence one method for making such assessments. Specifically, by using active D-DHA as a marker compound given at a specific fixed dose for a sufficient period of time to establish a steady state concentration in blood which is based on that dose, the amount of DHA supplied by the patient's diet can now be assessed by combining the ratio of active D-DHA to DHA established by an assay and then correlating the amount of DHA based on the amount of active D-DHA generated by the fixed dose and the ratio of deuterated DHA in the blood.

However, at the start of therapy, the attending clinician can only approximate the amount of DHA consumed by the patient based on a detailed analysis of the patient's diet and assuming that the reported diet accurately reflects wbat the patient is, in fact, consuming. Given the uncertainty and the effort required, the compositions and methods provided herein start from a different perspective-one where the approximate average daily consumption rate of DHA is not relied upon. Rather, the clinician starts with a dose of D-DHA that is sufficient to capture the average daily dose of DHA consumed by about 90% of patients, or about 95% of patients or about 99% or more of patients. Specifically, the clinician can initiate therapy with about 150 mg/day of D-DHA (about 90% or more of patients), or about 200 or 250 mg/day of D-DHA (about 95% or more of patients), or about 500 mg/day of D-DHA to further increase the percentage of captured patients), or about 1,000 mg/day to capture about 99% or more of the patients to be treated.

With this approach, the clinician can then evaluate the average amount of DHA consumed per day in the patient by testing a blood sample for the concentration of DHA found therein after a steady state concentration of active D-DHA has been reached in the blood. Once this amount is determined, the clinician can either maintain the dose of D-DHA or adjust that dose to an amount that would provide for a target ratio of active D-DHA to DHA that would be therapeutic. In addition and as noted above, the clinician can target the dose to a level that exceeds a therapeutic ratio of active D-DHA to DHA in order to provide for accountable days of non-compliance by the patient.

In addition, such testing can assess whether the patient is being compliant with dosing instructions provided by the attending clinician. In some embodiments, when the blood tests indicate that the patient is not achieving target concentrations of active D-DHA, the methods described herein include restricting the patient's consumption of dietary DHA (e.g., to no more than about 100 mg/day) during therapy with D-DHA or increasing the dose of D-DHA or both. In some embodiments, the method includes restricting the patient's consumption of dietary DHA to no more than about 70 mg/day during therapy with D-DHA. In some embodiments, the method includes restricting the patient's consumption of dietary DHA to no more than about 60 mg/day during therapy with D-DHA. In some embodiments, the method includes restricting the patient's consumption of dietary DHA to no more than about 50 mg/day during therapy with D-DHA. In some embodiments, the consumption of dietary DHA is restricted to no more than about 50 mg to about 130 mg per day or to no more than about 60 mg to about 120 mg per day, or no more than about 60 mg to about 110 mg per day.

The actual methodology of testing for active D-DHA concentration and the ratio of active D-DHA to DHA in a blood sample is not critical as long as the test analysis is conducted using a fixed dosage of D-DHA over at least a 14 day period and then testing is done with the patient's plasma based on a patient having fasted for at least 8 hours prior to the blood draw. Testing for active D-DHA in red blood cells does not require fasting but will require a longer period of time prior to reaching steady state conditions in these cells (typically at least about 6 weeks after the start of treatment).

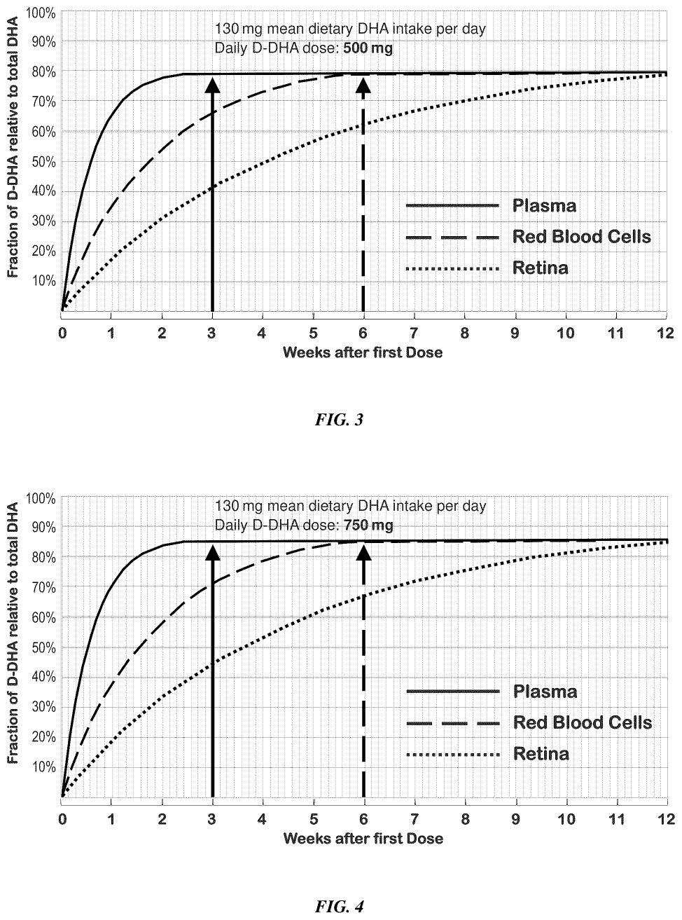

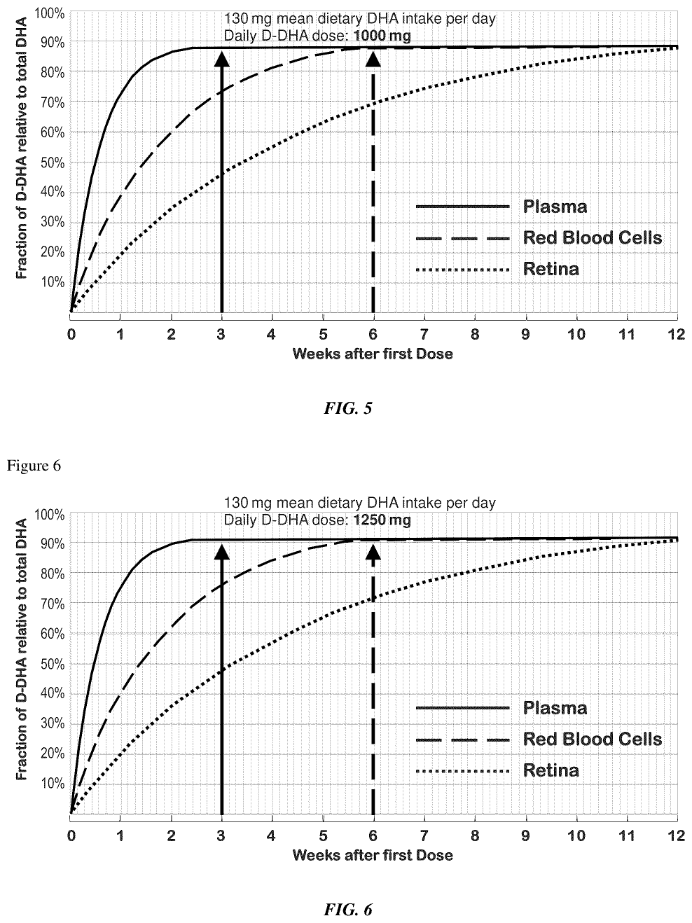

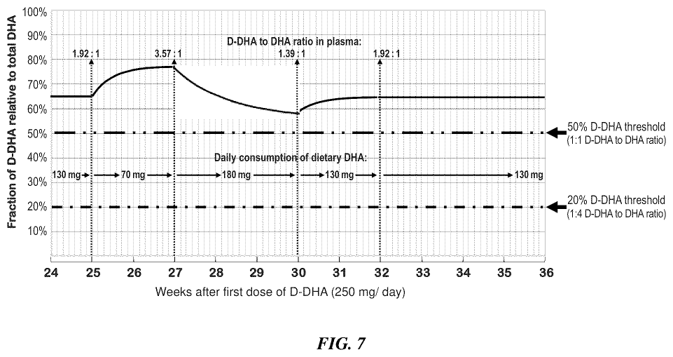

In addition to the above, standardized curves to establish timing and concentrations to reach steady state in blood for different dosing regimens for D-DHA can be generated for each dose of DHA (see FIGS. 3, 4, 5, and 6). As shown in the Examples, standardized curves for determining the time between initiation of therapy and when steady state concentrations are provided for two different markers-blood plasma and RBCs using two different dosing regimens. Using such procedures, a standardized curve for any dosing concentration of D-DHA can be established.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims.

As used herein, the following abbreviations have the following definitions. Terms that are not defined have their accepted scientific definitions.
AMD=age-related macular degeneration
DHA=docosahexaenoic acid
D-10 DHA=a DHA composition having on average DHA compounds comprising a total deuteration of at least about 80 percent at the bis-allylic sites (replacing 80% of the hydrogen atoms at these sites with deuterium atoms) and about 30% or less of total deuteration at the two mono-allylic sites (about 30% or less of the 4 hydrogen atoms at the two mono-allylic sites have been replaced with deuterium).

mg=milligrams

### Example 1

### Preparation of Deuterated Docosahexaenoic Acid Ethyl Ester

Following the procedure of U.S. Pat. No. 10,730,821, a composition comprising docosahexaenoic acid ethyl ester was prepared which was deuterated at the bis-allylic positions at a level of greater than 80% on average and at the mono-allylic positions at less than 35% on average. The specifics for the deuteration are set forth in formula (I) (as set forth above and reproduced below):

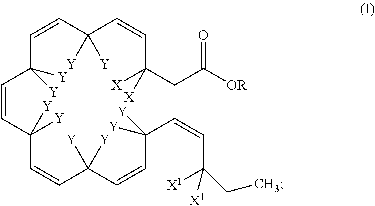

where R is hydrogen, a C₁-C₁₀ alkyl or a pharmaceutically acceptable salt;

each X is independently hydrogen or deuterium wherein the aggregate amount of the amount of deuterium defined by both X groups is such that, on average, the total amount of deuteration at the carbon atom is less than about 5%;

each X¹ is independently hydrogen or deuterium wherein the aggregate amount of the amount of deuterium defined by both X¹ is such that, on average, less than about 25% of the X¹ groups are deuterium and the remainder are hydrogen;

each Y is independently hydrogen or deuterium wherein the specific value for each Y is selected such that on average:
a) from about 87 to about 92 percent of the Y groups on each carbon atom are deuterium;
b) from more than about 5 about to 12 percent of the Y groups on each carbon atom are substituted with a single hydrogen and a single deuterium; and
c) less than about 2 percent of the Y groups of each carbon atom are substituted with two hydrogen atoms;

provided that the sum of all Y groups equal 10.

### Example 2

### Method to Reduce the Rate of Disease Progression

It has been established that oxidative stress plays a central role in AMD. Iron, a potent generator of hydroxyl radicals through the well-known Fenton reaction, has been implicated in AMD. DHA is the most abundant PUFA in the photoreceptor membranes of the retina and the bis-allylic positions are readily oxidized producing toxic oxidation products such as carboxyethylpyrrole adducts, which increase in the retina of AMD patients.

In this example, two cohorts of adult male wild-type C57BL/6J mice (Jackson Labs) were used. Both cohorts were fed identical feed containing 0.25% or 0.5% D-DHA or matching amounts of DHA as controls. After 4 weeks, the mice were given an intravitreal injection of 1 microliter of 0.5 millimolar ferric ammonium citrate diluted in 0.9% saline (test) or 1 microliter of 0.9% saline (control). Mice were then continued on their respective diets for 4 weeks after injection in order to evaluate whether D-DHA could protect against geographic atrophy which is related to AMD.

Afterwards, BAF and IRAF images displayed hypo-AF in the superior retinas of test mice, similar to geographic atrophy. On the other hand, the test mice were fully protected against geographic atrophy when the concentration of D-DHA was at least 50% of the total DHA in the retina. At lower concentrations of D-DHA to DHA, protection was achieved but at reduced rates that correlated to the relative concentration of D-DHA (lower concentrations of D-DHA provided less protection).

These results evidence that D-DHA inhibits oxidation at the bis-allylic sites which oxidation is a required component of AMD. Still further, these results evidence that at ratios of about 1:4 of D-DHA to DHA, the protection is accorded whereas at ratios of at least about 1:1 D-DHA to DHA protect is maximal. Liu, et al., Aging Cell, 2022 provides complete experimental for this Example and is incorporated herein by reference in its entirety.

Based on the above experiment, the following provides a proposed evaluation in reduction in the rate of macular degeneration progression in a cohort of patients treated with deuterated docosahexaenoic acid ethyl ester, as compared to a cohort of test patients treated with placebo. Specifically, the treated cohort is administered 250 mg/day of deuterated docosahexaenoic acid ethyl ester or 250 mg/day of safflower oil. The patients are maintained on this dosing regimen throughout the clinical study. Periodic measurements of further geographic atrophy development are obtained.

Dosing is continued for 6 or 12, or 18 or 24 months. At that time, the average extent of geographic atrophy progression is measured for each cohort. The efficacy of the treatment protocol is evaluated by comparing the extent of geographic atrophy progression in a treated population against a placebo population. Specifically, the methods describe herein provide a statistically significant reduction in the rate of disease progression.

### Example 3

### Determining the Reduction pf Disease Progression

In this example, the reduction in disease progression can be determined as follows:
a) determine the average rate of disease progression for a cohort of patients treated with the deuterated docosahexaenoic acid ethyl ester by measuring the extent of geographic atrophy in each of the patient's retina at the start of therapy and at 6, 12, 18 or 24 months post start of therapy, determining the difference between the extent of atrophy at the start of therapy and at the later time point, and then obtaining an average for that differential and assigning a first value designated as “A” to that average differential;
b) determine the average rate of disease progression for a cohort of patients treated with placebo (e.g., safflower oil) by measuring the extent of geographic atrophy in each of the patient's retina at the start of therapy and at 12, 18 or 24 months post start of therapy, determining the difference between the extent of atrophy at the start of therapy and at the later time point, and then obtaining an average for that differential and assigning a second value designated as “B” to that average differential;
c) calculating the difference between B and A and assign “C” to that value (e.g, B−A=C);
d) assign a positive value to “C” if B is greater than A;
e) assign a negative value to “C” if B is less than A; and
f) divide C by B and multiply by 100 [(C/B)×100].

As per this example, treated patients will have a positive percent reduction in geographic atrophy area that is statistically significant and preferably at least a 20 to 25 percent reduction. That is to say that if A has an arbitrary value of 45 and B has an arbitrary value of 60, then B−A=C gives a value for C of 15. Then dividing C/B gives 15/60 and multiplying that value by 100=25%.

### Example 4

### Determination In The Reduction In Disease Progression

Alternatively, the rate of disease progression for an individual patient with an oxidative retinal disease can be assessed by the following:
a) determine the rate of disease progression for the individual patient treated with 1,000 mg of D-DHA by measuring the extent of geographic atrophy in the patient's retina at the start of therapy and at 6, 12, 18 or 24 months post start of therapy and assigning a third value “D” to that differential;
b) determine the average rate of disease progression for a cohort of patients treated with placebo (safflower oil) by measuring the extent of geographic atrophy in each of the patient's retina at the start of therapy and at 6, 12, 18 or 24 months post start of therapy, determining the difference between the extent of atrophy at the start of therapy and at the later time point, and then obtaining an average for that differential and assigning a second value designated as “E” to that average differential;
c) calculating the difference between D and E and assign “F” to that value (e.g, E−D=F);
d) assign a positive value to “F” if E is greater than D;
e) assign a negative value to “F” if E is less than D; and
f) divide F by E and multiply by 100 [(F/E)×100].

As per this example, a treated patient will have a positive percent reduction in geographic atrophy that is statistically significant and preferably at least a positive 20 percent reduction. That is to say that if D has an arbitrary value of 50 and E has an arbitrary value of 75, then E−D=F gives a value for F of 25. Then dividing F/E gives 50/100 and multiplying that value by 100=33.3%.

Methods for calculating disease progression in oxidative neurological diseases are well known including those set forth in U.S. Pat. No. 11,351,143 as well as in U.S. Ser. No. 17/408,285 both of which are incorporated herein by reference in their entirety.

### Example 5

### Determination of the D-Dha Levels

Mature adult C57BL/6J mice were fed for 77 days with a customized rodent diet containing 0.5% w/w D-DHA and no natural DHA. The animals were sacrificed, and plasma, red blood cells and retinal tissues dissected at study Days 8, 19, 38, 77, (six animals per time point, 3 males +3 females). Active D-DHA +DHA were extracted from samples and derivatized into methyl esters with a mixture of heptane/toluene (63:37 by volume), and methanol/dimethoxypropane/sulfuric acid (85:11:4 by volume) by gentle shaking at 80° C. for 2 hours, followed by separation and drying down of the organic phase under nitrogen. D-DHA+DHA methyl esters were structurally identified and quantified by gas chromatography coupled with a tandem mass spectrometry detector and D-DHA substitution levels (in percent of total DHA) were calculated. The results are represented in Table 3 and graphed in FIG. 1. The measured data revealed that the active D-DHA substitution rates follow a first order kinetics, i.e., they increase by 50% of the prior measured values at regular intervals (hereinafter called accretion half life) in each sample type (about every 2.7 days in plasma, about every 8.75 days in red blood cells, and about every 23.42 days in retina whereby the maximum concentration (steady-state) is reached earlier in plasma and red blood cells (which are accessible in human subjects by a simple blood draw) than in retina (which is not accessible in living human subjects). With known accretion half-lives, the steady-state concentrations can be calculated by single and/or multipoint measurements without the need to wait until steady-state is actually reached.

TABLE 3
D-DHA accretion in murine tissues
D-DHA substitution levels (in percent of total DHA) at
multiple time points after first exposure

| | Sampling (Days) | | | | |
|---|---|---|---|---|---|
| | 0 | 8 | 19 | 38 | 77 |
| Plasma | 0 | 84.78% | 94.17% | 97.34% | 98.43% |
| Red Blood Cells | 0 | 48.24% | 72.84% | 94.12% | 96.65% |
| Neuroretina | 0 | 20.89% | 42.94% | 70.74% | 92.10% |

### Example 6

### Predicting Retinal D-Dha Concentrations

In contrast to controlled experimental diets, naturally occurring DHA is consumed by patients treated with D-DHA which can dilute the relative percentage of administered D-DHA absorbed with the total DHA pool and its final concentration at steady state in blood and eventually in the target tissue such as retina. The data and standard curves referred to above allow for the determination of retinal steady-state active D-DHA concentrations with reasonable accuracy by calculating the D-DHA/total DHA ratio in plasma and/or red blood cells. Table 4 exemplifies this by using a mean daily dietary intake of about 130 mg of DHA per day (which represents the 90^{th} percentile of the mean usual DHA intake by males >51 years of age in the US.

FIGS. 3, 4, 5, and 6 illustrate how measuring plasma and red blood cell active D-DHA concentrations can be used to predict expected retinal steady-state concentrations. With a known fixed daily dose and the measured of active D-DHA substitution rate at steady-state, this also allows to approximate and monitor the mean dietary DHA intake of individual patients and permits either a timely adjustment of the daily D-DHA dose or dietary interventions to reduce natural DHA intake until the desired therapeutic active D-DHA substitution levels, preferably about 50% or more, can be reached.

TABLE 4
Example for D-DHA steady-state
substitution rates at 3 different fixed daily
D-DHA doses at a given dietary background intake of natural DHA

| Fixed daily D-DHA dose | 250 mg | 500 mg | 1000 mg |
|---|---|---|---|
| DHA in human diet | 130 mg | 130 mg | 130 mg |
| (example: 130 mg/day) | | | |
| Total daily DHA intake | 380 mg | 630 mg | 1130 mg |
| (D-DHA + DHA) | | | |
| D-DHA substitution rate at | 66% | 79% | 88% |
| steady-state (% of total DHA) | | | |
| Ratio of D-DHA to DHA | ~1.9:1 | ~3.8:1 | ~7.7:1 |

In each case, the first order kinetics evidence that a dose of 250 mg dose of D-DHA will provide for a steady-state ratio of active D-DHA based on the total DHA concentration present, as set forth in Table 4. Extrapolating backwards from the case in Table 4, a dose of at least 130 mg/day of D-DHA should provide a 1:1 ratio of active D-DHA to DHA. That ratio has been shown above to be therapeutic. And a dose of 250 mg of higher should provide a ratio of 1.9:1 or higher which provides a margin for non-compliance as decribed herein above.

### Example 7

FIG. 7 is intended to illustrate the variation in the steady state ratio of D-DHA to DHA based on a hypothetical patient dosed at 250 mg of D-DHA ethyl ester per day and consuming on average about 130 mg of DHA or less per day. As illustrated in FIG. 7, the daily ratio of active D-DHA to DHA in that patient will fluctuates at steady state due to normal increases and decreases in the amount of DHA and D-DHA consumed/administered coupled with whatever percent is absorbed. This is evidenced by the non-uniformity in the curve.

As to specifics, at about 24 to 25 weeks from the start of therapy, the patient is confirmed to be at a steady state concentration of active D-DHA relative to DHA in all major body tissues including plasma, liver and the skeletal muscle tissue. The active D-DHA plasma levels are about 66% and the ratio of D-DHA to DHA in the plasma is about 1.92:1 as is predicted.

After at the end of week 25, the patient reduces the consumption of dietary DHA to about 70 mg of DHA per day for 2 weeks. Thereafter, the patient increses the consumption of dietary DHA to about 180 mg of DHA per day for 3 weeks, and then resumes the prior consumption of DHA at about 130 mg/day. As the ratio of active D-DHA to DHA at the start of therapy is about 1.92:1 (about 66% active D-DHA plasma levels), there is an increase in ratio of active D-DHA to DHA due to the reduced amount of DHA consumed that increases the relative percentage of active D-DHA as a function of the entirety of DHA consumed to about 78% after 2 weeks. This is evidenced by an increase in the active D-DHA to DHA) ratio to about 3.6:1. A subsequent increased intake in DHA leads to a reduction of active D-DHA plasma levels to about 58% afrer 3 weeks which equals an active D-DHA to

DHA ratio of about 1.4:1. Thereafter, the patient returned to consuming about 130 mg/day of DHA which resulted in a return to 66% active D-DHA plasma levels two week later with an active D-DHA to DHA ratio of 1.92:1. This is illustrated in FIG. 7 where, again, the fluctuation in the curve evidences changes in the amount of DHA consumed.

The same periodic monitoring scheme will reveal instances where the active D-DHA levels have fallen below intended therapeutic thresholds, fore example below 59% or below 20% D-DHA.

Tables 5, 6, and 7 are based on Table 2 and describe the time it takes for plasma D-DHA levels to fall below 50% or below 20% with 5 different daily doses of D-DHA in (i) patients in the 90^{th} percentile of daily intake consuming up to 130 mg DHA (Table 5), (ii) those in the 95^{th} percentile with a daily intake of up to 180 mg DHA (Table 6), and (iii) an example of a patient consuming more than one meal of oily fish per week with up to 260 mg daily DHA intake.

TABLE 5
90^{th} percentile DHA background diet (US males*):

| | | | | | |
|---|---|---|---|---|---|
| Daily D-DHA Dose | 250 mg | 500 mg | 750 mg | 1000 mg | 1250 mg |
| Background DHA/Day | 130 mg | 130 mg | 130 mg | 130 mg | 130 mg |
| Total DHA/Day | 380 mg | 630 mg | 880 mg | 1130 mg | 1880 mg |
| D-DHA Substitution Rate | ~66% | ~79% | ~85% | ~88% | ~91% |
| D-DHA/DHA Ratio | 1.92:1 | 3.85:1 | 5.77:1 | 7.69:1 | 9.62:1 |
| Time to reach ≤50% in Plasma | up to | 4 up to | up to | up to | up to |
| (1:1 D-DHA to DHA Ratio) | 22 days** | 6 days | 60 days | 70 days | 77 days |
| w/o taking oral D-DHA | | | | | |
| Time to reach 20% in Plasma | up to | up to | up to | up to | up to |
| (0.2:1 D-DHA to DHA Ratio) | 77 days | 46 days | 101 days | 125 days | 132 days |
| w/o taking oral D-DHA | | | | | |

TABLE 6
95^{th} percentile DHA background diet (US males*):

| | | | | | |
|---|---|---|---|---|---|
| Daily D-DHA Dose | 250 mg | 500 mg | 750 mg | 1000 mg | 1250 mg |
| Background DHA/Day | 180 mg | 180 mg | 180 mg | 180 mg | 180 mg |
| Total DHA/Day | 430 mg | 680 mg | 930 mg | 1180 mg | 1430 mg |
| D-DHA Substitution Rate | ~58% | ~74% | ~81% | ~85% | ~87% |
| D-DHA/DHA Ratio | 1.39:1 | 2.78:1 | 4.17:1 | 5.56:1 | 6.94:1 |
| Days to reach ≤50% in Plasma | up to | up to | ~up to | ~up to | ~up to |
| (1:1 D-DHA to DHA Ratio) | 11 days** | 35 Days | 49 Days | 59 Days | 67 Days |
| w/o taking oral D-DHA | | | | | |
| Time to reach 20% in Plasma | up to | up to | up to | up to | up to |
| (0.2:1 D-DHA to DHA Ratio) | 66 Days | 90 Days | 104 Days | 114 Days | 121 Days |
| w/o taking oral D-DHA | | | | | |

TABLE 7
3) Example of high DHA background diet in patients
consuming >1 meal of oily fish per week:

| | | | | | |
|---|---|---|---|---|---|
| Daily D-DHA Dose | 250 mg | 500 mg | 750 mg | 1000 mg | 1250 mg |
| Background DHA/Day | 260 mg | 260 mg | 260 mg | 260 mg | 260 mg |
| Total DHA/Day | 510 mg | 760 mg | 1010 mg | 1260 mg | 1510 mg |
| D-DHA Substitution Rate | ~49% | ~66% | ~74% | ~79% | ~83% |
| D-DHA/DHA Ratio | 0.96:1 | 1.92:1 | 2.88:1 | 3.85:1 | 4.81:1 |
| Days to reach ≤50% in Plasma | already | up to | up to | up to | up to |
| (1:1 D-DHA to DHA Ratio) | below | 8 days | 26 days | 38 days | 47 days |
| w/o taking oral D-DHA | 50% | | | | |
| Time to reach 20% in Plasma | up to | up to | up to | up to | up to |
| (0.2:1 D-DHA to DHA Ratio) | 29 days | 63 days | 81 days | 93 days | 102 days |
| w/o taking oral D-DHA | | | | | |

* USDA, Agricultural Research Service, 2021 “Usual Nutrient Intake from Food and Beverages, by Gender and Age, What We Eat in America - http://www.ars.usda.gov/nea/bhnrc/fsrg

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for assessing the average daily consumption of DHA in a patient comprising:
a) administering a fixed daily amount of D-DHA to the patient for a period of time sufficient to reach steady state concentrations in the patient's blood or a component thereof;
b) obtaining a sample of the patient's blood or a component thereof and assessing the amount of active D-DHA in the sample based on the fixed daily amount of D-DHA administered as well as the ratio of active D-DHA to DHA;
c) correlating the ratio of active D-DHA to DHA with the amount of D-DHA found in the blood or a component thereof to obtain a value of DHA in the blood; and
d) adjusting the dose of D-DHA in response to the value of DHA in the blood.

2. The method of claim 1, wherein the blood component tested is plasma and the fixed daily amount of D-DHA administered to the patient reaches a steady state concentration in the plasma in about 14 days.

3. The method of claim 1, wherein the blood component tested is a population of red blood cells and the fixed daily amount of D-DHA administered to the patient reaches a steady state concentration in these cells in about 45 days.

4. A method for treating an oxidative neurodegenerative or retinal disease in the patient comrprising:
a) administering a fixed daily amount of D-DHA to the patient for a period of time sufficient to reach steady state concentrations in the patient's blood or a component thereof;
b) obtaining a sample of the patient's blood or a component thereof and assessing the amount of active D-DHA in the sample based on the fixed daily amount of D-DHA administered as well as the ratio of active D-DHA to DHA;
c) correlating the ratio of active D-DHA to DHA with the amount of active D-DHA found in the blood or a component thereof to obtain a value of DHA in the blood;
d) adjusting the amount of the fixed daily dose of D-DHA as necessary to achieve a therapeutically effective amount; and
e) continuing the adjusted or maintained fixed daily amount of D-DHA in order to treat the disease.

5. The method of claim 4, wherein the patient is subjected to successive blood tests to confirm that the therapeutically effective amount being maintained.

6. The method of claim 5, wherein the successive blood tests are conducted monthly, quarterly, semi-annually, annually or at any interval deemed appropriate by the attending clinician.

7. The method of claim 4, wherein the oxidative neurodegenerative disease is Amyotrophic Lateral Sclerosis (ALS), Jacobson Syndrome, spinal muscular atrophy, multiple system atrophy, Alzheimer's Disease (AD) and mild cognitive impairment, Huntington's Disease, infantile neuroxonal disease (INAD), Parkinson's Disease, or Progressive supranuclear palsy (PSP).

8. The method of claim 4, wherein the oxidative retinal disease is wet and dry age-related macular degeneration (including geographic atrophy associated therewith), retinal degeneration, cataracts, retinitis pigmentosa, diabetic retinopathy, glaucoma, or Stargardt Disease.

9. A pharmaceutical composition comprising from about 100 mg to about 1,250 mg of D-DHA in one or more pharmaceutically acceptable units for daily administration and optionally in the presence of a pharmaceutically acceptable carrier.

10. The pharmaceutical composition of claim 9, wherein the composition comprises from about 200 to about 1,000 mg of D-DHA for daily administration in one or more pharmaceutically acceptable units.

11. The pharmaceutical composition of claim 9, wherein the composition comprises at least about 250 mg of D-DHA for daily administration in one or more pharmaceutically acceptable units.

12. The pharmaceutical composition of claim 9, wherein the composition comprises at least about 500 mg of D-DHA for daily administration in one or more pharmaceutically acceptable units.

13. The pharmaceutical composition of claim 9, wherein the composition comprises at least about 1,000 mg of D-DHA for daily administration in one or more pharmaceutically acceptable units.

14. The pharmaceutical composition of any one of claims 9, wherein the composition provides at least 4 days per month of accountable medication non-compliance days.

15. The pharmaceutical composition of claim 14, wherein the composition provides at least 7 days per month of accountable medication non-compliance days.

16. A method for reducing a rate of disease progression for an oxidative retinal or neurodegenerative disease in a patient, the method comprising periodic administration to the patient of a composition comprising deuterated docosahexaenoic acid or ester thereof of formula (I): 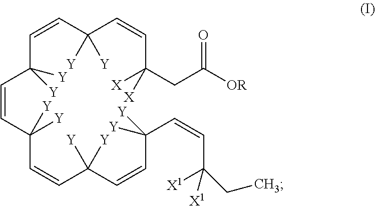
where R is hydrogen, a C₁-C₁₀ alkyl or a pharmaceutically acceptable salt;
each X is independently hydrogen or deuterium wherein the aggregate amount of the amount of deuterium defined by both X groups is such that, on average, the total amount of deuteration at the carbon atom is less than about 5%;
each X¹ is independently hydrogen or deuterium wherein the aggregate amount of the amount of deuterium defined by both X¹ is such that, on average, less than about 25% of the X¹ groups are deuterium and the remainder are hydrogen;
each Y is independently hydrogen or deuterium wherein the specific value for each Y is selected such that on average:
a) from about 87 to about 92 percent of the Y groups on each carbon atom are deuterium;
b) from more than about 5 about to 12 percent of the Y groups on each carbon atom are substituted with a single hydrogen and a single deuterium; and
c) less than about 2 percent of the Y groups of each carbon atom are substituted with two hydrogen atoms;
provided that the sum of all Y groups equal 10.
